(19) United States
(12) Patent Application Publication
(10) Pub. No.: US 2026/0174324 A1
(43) Pub. Date: Jun. 25, 2026

(54) VISUAL FIELD ESTIMATION APPARATUS, NEURAL NETWORK MANUFACTURING METHOD, AND PROGRAM

(71) Applicant: Makoto KOYAMA, Chiba (JP)

(72) Inventor: Makoto KOYAMA, Chiba (JP)

(21) Appl. No.: 19/111,800

(22) PCT Filed: Sep. 16, 2022
(86) PCT No.: PCT/JP2022/034831

§ 371 Date: Feb. 20, 2026

(51) Int. Cl. A61B 3/024; G16H 50/20

(52) U.S. Cl. CPC A61B 3/024; G16H 50/20

(57) ABSTRACT

Provided is a visual field estimation apparatus using a machine learning model that has been machine-trained so that, with respect to a plurality of eyes to be examined, at least one of image information of retina and three-dimensional structure information of retina is acquired at each of a plurality of time points, visual field related information relating to a visual field of the eye to be examined is acquired at a corresponding time point, at least one of the image information of the retina and the three-dimensional structure information of the retina regarding each of the eyes to be examined is input to the machine learning model as input information, visual field change information representing change of the visual field and obtained based on the visual field related information at the plurality of time points with regard to the corresponding eye to be examine is used as training information, and visual field change information estimated on the basis of the input information is output from the machine learning model.

### TECHNICAL FIELD

The present disclosure relates to a visual field estimation apparatus, a neural network manufacturing method, and a program.

### BACKGROUND ART

Estimating the progress of visual field defects caused by glaucoma, etc., is important for determining a therapeutic method to retard the progression thereof. Conventionally, for assessing the progression rate, visual field measurement is performed at least 6 times at certain intervals, an MD value (Mean Deviation: weighted mean deviation of a patient's vision at all measurement points arranged on the entire visual field a normal person, from a normal person's vision of the same age as the patient) is obtained at each measurement, and a time-series regression line of the MD values is obtained for estimating the progression rate.

### PRIOR ART

### Patent Document

Patent Document 1: Japanese Unexamined Patent Publication (Kokai) No. 2020-121012

### SUMMARY

However, according to the above-mentioned conventional estimation method of the progression rate, a large amount of time is required until the estimation of the progression rate becomes possible. For example, if the measurement is performed once in 6 months, two years and a half is required until 6 times of measurement is performed. Further, if the visual field defect becomes close to the center of the eye of a patient, 24-degree visual field measurements and 10-degree visual field measurements are often performed alternately. In this case, it takes more than 5 years until 6-times of 24-degree visual field measurement data and 6-times of 10-degree visual field measurement data are obtained.

As above, according to the method of the prior arts, at least two years are required, and the visual field defect may progress during the two years. On the other hand, it is known that administration of early and adequate treatment can effectively regard the progression of the visual field defects. Namely, performing more accurate assessment of the progression rate at an early stage is preferable, but actually, such a technology has not been thought of.

Patent Document 1 discloses an ophthalmologic visual field estimation apparatus provided with an acquisition unit which acquires at least one of the three-dimensional structure information and the front image of the retina of the eye to be examined, and a generation unit which generates estimated visual field information from at least one of the three-dimensional structure information and the front image, using a learned model. According to the technology disclosed in Patent Document 1, a visual field measurement test can be performed more easily. However, Patent Document 1 fails to consider the early estimation of the progression rate of the visual field defects.

The present disclosure has been considered in view of the above drawbacks, and one of the objectives of the present disclosure is to provide a visual field estimation apparatus, a neural network manufacturing method, and a program capable of performing early assessment of the progression rate of the visual field defects.

In order solve the drawbacks of the conventional examples, one aspect of the present disclosure is a visual field estimation apparatus comprising: a retention device which retains a machine learning model that has been machine-trained so that, with respect to a plurality of eyes to be examined, at least one of image information of retina and three-dimensional structure information of retina is acquired at each of a plurality of time points, visual field related information relating to a visual field of the eye to be examined is acquired at a corresponding time point, at least one of the image information of the retina and the three-dimensional structure information of the retina regarding each of the eyes to be examined is input to the machine learning model as input information, visual field change information representing change of the visual field and obtained based on the visual field related information at the plurality of time points with regard to the corresponding eye to be examine is used as training information, and visual field change information estimated on the basis of the input information is output from the machine learning model; an acquisition device which acquires at least one of image information of retina and three-dimensional structure information of retina of an eye to be examined of a person undergoing an eye examination; an estimation device which inputs the image information of the retina or the three-dimensional structure information of the retina of the eye to be examined of the person undergoing the eye examination, acquired by the acquisition device, to the machine learning model retained in the retention device, and obtains the output from the retention device as an estimation value of change of the visual field related information; and an output device which outputs the estimation value obtained by the estimation device for subjecting to a predetermined process.

According to the present disclosure, the progression rate of visual field defects can be accessed early.

### BRIEF DESCRIPTION OF DRAWINGS

FIG. 1 is a block diagram representing a configuration example of a visual field estimation apparatus according to an aspect of the present disclosure.

FIG. 2 is a block diagram representing a configuration example of a machine learning model used by a visual field estimation apparatus according to an aspect of the present disclosure.

FIG. 3 is a functional block diagram representing an example of a visual field estimation apparatus according to an aspect of the present disclosure.

FIG. 4 is an explanatory view showing an example of a display output by a visual field estimation apparatus according to an aspect of the present disclosure.

FIG. 5 is a block diagram representing a configuration example of β-VAE used by a visual field estimation apparatus according to an aspect of the present disclosure.

### ASPECTS OF DISCLOSURE

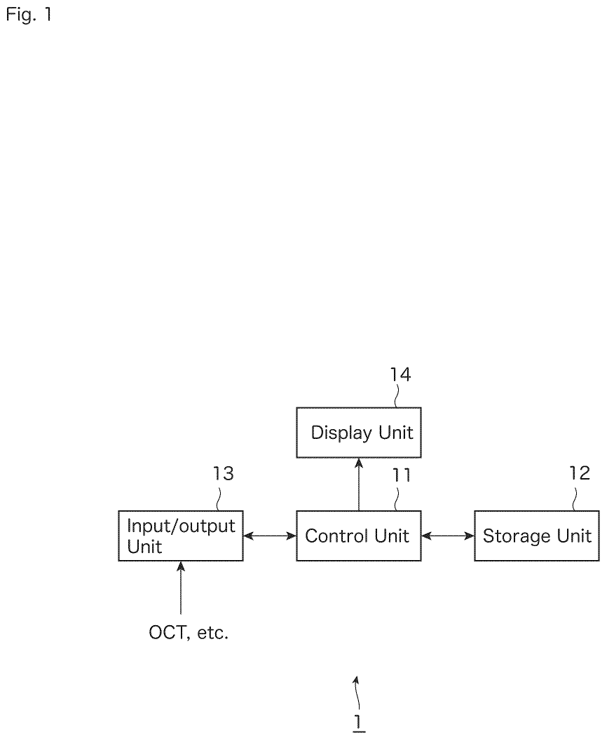

An aspect of the present disclosure will be explained with reference to the drawings. A visual field estimation apparatus 1 according to an aspect of the present disclosure can be realized by using an ordinary computer. As exemplified in FIG. 1, the visual field estimation apparatus 1 comprises a control unit 11, a storage unit 12, an input/output unit 13, and a display unit 14.

### Hardware Configuration

The control unit 11 is a control device such as a processor, etc., operating in accordance with a program, and the control unit 11 operates in accordance with a program stored in the storage unit 12. According to an example of the present aspect, the storage unit 12 functions as a retention device which retains a machine learning model having been machine-trained to estimate and output at least visual field change information on the basis of input information, the input information being at least one of image information of retina and three-dimensional structure information of retina.

Then, the control unit 11 acquires at least one of image information or three-dimensional structure information of the retina of an eye to be examined of a person undergoing an eye examination, inputs the acquired image information or three-dimensional structure information of the retina of the eye to be examined of the person undergoing an eye examination to the machine learning model retained in the storage unit 12, and obtains the output therefrom as an estimation value for the change of the visual field related information. Then, the control unit 11 outputs the estimation value for the change of the visual field related information. Details of these operations of the control unit 11 will be described later.

The storage unit 12 includes a memory device and a disk device, functions as the above-mentioned retention device, and also retains a program to be executed by the control unit 11. The program may be provided by being stored in a computer readable and non-transitory storage medium and copied in the storage unit 12. Further, the storage unit 12 also operates as a work memory of the control unit 11.

The input/output unit 13 includes an input device such as a keyboard, etc. The input/output unit 13 receives information input in accordance with instructions by a user, and outputs the information to the control unit 11. For example, the input/output unit 13 is provided with an interface such as USB (Universal Serial Bus), etc., receives input of such as three-dimensional structure information of the retina output from an OCT (Optical Coherence Tomography) examination device, image information of the retina obtained by a fundus camera, and the like, through the interface, and outputs the received information to the control unit 11.

Furthermore, the input/output unit 13 may be provided with an interface (network interface, etc.) which outputs information to an external device, etc., in accordance with an instruction input from the control unit 11.

The display unit 14 is a display device, etc., which displays and outputs information in accordance with an instruction from the control unit 11.

### Terms

Here, simple definitions of the terms used in the explanation below are described.

### (1) Measurement Point

A measurement point is a point corresponding to the position of each of a plurality of optotypes arranged in a predetermined angle range from a fixation point, in an HFA (Humphrey Field Analyzer), etc. A measurement value at a measurement point is a brightness threshold value (for example, the lowest brightness value that can be visually recognized) of an optotype which can be visually recognized by a person undergoing an eye examination (patient). Hereinbelow, the simple expression of “threshold value” refers to this threshold value.

### (2) MD Value

An MD value (Mean Deviation) is a mean deviation which is a weighted mean deviation between measurement values of a person having a normal field of view and being the same age as a patient, and measurement values of the patient, at all measurement point.

### (3) MD Slope

An MD slope (MD Slope) is a slope of time-series MD values (temporal change), such as a slope of a regression line of MD values at a plurality of time points.

### (4) VFI Value

A VFI value (Visual Field Index) is an index in a visual field of 24 degrees around a fixation point, and is an index showing an abnormal level of a visual field with the central 4 points being largely weighted.

### (5) TD Value

A TD value (Hemi Field MD) is a total deviation between the upper-half visual field and the lower-half visual field.

### (6) Visual Field Index

A visual field index refers to a measurement result obtained by mixing:
a central 24-degree MD value,
a central 10-degree MD value,
a central 24-degree VFI value,
a central 24-degree TD value,
a central 10-degree TD value, etc. (or may be other range), around the fixation point,
and further, a central 24-degree visual field index and a central 10-degree visual field index measured by 24plus(1), 24plus(2) (https://www.crewt.co.jp/product/imo), or, 24-2C SITA Faster (https://www.zeiss.co.jp/meditec/product-portfolio/perimetry/humphrey-field-analyzer-3.html), and the like. In the explanation hereinbelow, an example using the central 24-degree and the central 10-degree is explained. However, in the present aspect, the process regarding the visual field can be performed by using the above-mentioned 24plus(1), 24plus(2), or 24-2C SITA Faster. Further, the central 30-degree can be used.

### (7) Total Deviation

A total deviation (Total deviation) refers to a difference between a mean value of threshold values of persons in the same age as the patient, at each measurement point, and a threshold value of the patient at a corresponding measurement point.

### (8) Pattern Deviation

A pattern deviation is obtained based on the total deviation, by modifying the threshold value of the patient at each measurement point toward the normal threshold value, reducing the height difference of the entire visual field, and highlighting a local depression area. Even when the threshold value of the visual field is decreased as a whole, due to cataract, etc., local visual field defects of glaucoma can be easily found by the pattern deviation.

### [Machine Learning Processing]

Next, machine learning processing by a machine learning model used in a visual field estimation apparatus 1 of an example according to an aspect of the present disclosure will be explained. In an example according to an aspect of the present disclosure, the visual field estimation apparatus 1 executes the machine learning processing of the machine learning model. However, this is only an example, and the machine learning processing of the machine learning model can be executed by a computer other than the visual field estimation apparatus 1.

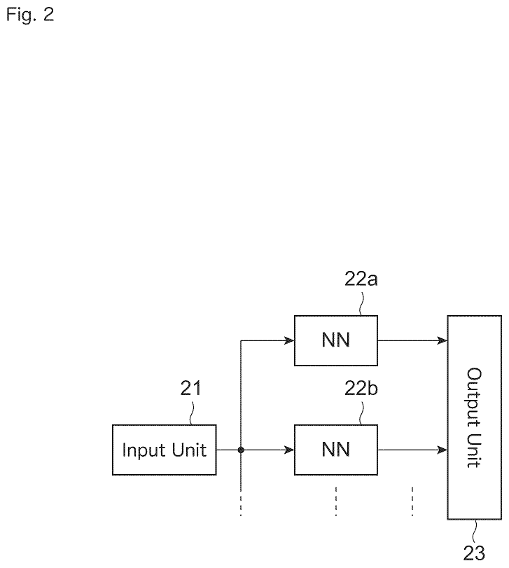

As exemplified in FIG. 2, the machine learning model 20 used by the visual field estimation apparatus 1 of the present aspect comprises an input unit 21, at least one neural network (NN) 22a, b . . . , and an output unit 23.

Each neural network 22a, b . . . , retains information regarding parameters (weight, bias, etc.) of a neural network, and generates output data on the basis of the below-mentioned input data input from the input unit 21, and the parameter. In an example of the present aspect, the output data from the neural network 22a, b . . . , includes visual field change information at each predetermined visual field range (for example, the central 24-degree, the central 10-degree, or a mixture of these).

Here, the visual field change information can be a slope value itself of a regression line representing the time-series change of the visual field index, such as a MD slope, etc., (threshold value in clinic), or can be a total deviation, or a pattern deviation. Such calculations in the neural network 22 (hereinbelow, when identifying each of the neural networks 22a, b . . . is not required, an expression “neural network 22” is used) are widely known, and thus, detail explanations therefor are omitted here.

According to an example of the present aspect, the neural networks 22a, b . . . can include the one corresponding to the left eye to be examined, and the one corresponding to the right eye to be examined. Namely, according to an example of the present aspect, the machine learning model 20 is provided with all of the following neural networks:
neural network receiving three-dimensional structure information of the left eye retina as input data, and outputting central 24-degree visual field change information of the left eye;
neural network receiving image information of the left eye retina as input data, and outputting central 24-degree visual field change information of the left eye;
neural network receiving three-dimensional structure information of the right eye retina as input data, and outputting central 24-degree visual field change information of the right eye;
neural network receiving image information of the right eye retina as input data, and outputting central 24-degree visual field change information of the right eye;
neural network receiving three-dimensional structure information of the left eye retina as input data, and outputting central 10-degree visual field change information of the left eye;
. . .
(in this case, eight neural networks in total, covering the difference of input data, difference of the left eye and the right eye, and the difference in the type of output data).

The input unit 21 receives input data which should be input to any one of the neural networks 22a, b, . . . , and outputs the input data to any one of the corresponding neural networks 22a, b, . . . , as input to any one of the neural networks 22a, b, . . . .

For example, the input unit 21 receives input of the input data, as well as information representing the type of the input data (whether the three-dimensional structure information of the retina, or the image information of the retina, or the like), and information representing whether the input data relates to the left eye or the right eye.

Then, on the basis of the information representing the type of the input data and the information representing whether the input data relates to the left eye or the right eye (hereinbelow, these are referred to as output destination selection information), the input unit 21 selects a neural network 22 to which the input data which has been input should be output. The output destination selection information may be associated with information representing a neural network 22 to which the output is destined, set in an information table, and stored in the storage unit 12, and the selection may be performed with reference to the settings.

The input unit 21 outputs the input data to the selected neural network 22. Here, the selected neural network 22 is not always one. For example, in case that the output destination selection information represents “three-dimensional structure information” and information relating to “the left eye”, the input unit 21 selects, from among the neural networks 22,
neural network receiving three-dimensional structure information of the left eye retina as input data, and outputting central 24-degree visual field change information of the left eye; and
neural network receiving three-dimensional structure information of the left eye retina as input data, and outputting central 10-degree visual field change information of the left eye; (namely, all of, or at least a part of the neural networks having the same type of input data), and outputs the input data to the selected neural networks.

The output unit 23 generates output data on the basis of the data output from the neural network 22 which has received the input data, from among the at least one neural network 22 provided in the machine learning model 20.

When the input data is input to only one neural network 22, the output unit 23 outputs the output data output from the only one neural network 22, as it is. Whereas, when the input data is input to a plurality of neural networks 22, the output unit 23 synthesizes the output data from the plurality of neural networks 22, and outputs the synthesized output data. Here, a method for the synthesis can be appropriately selected and adopted from a widely-known various methods for synthesizing out data from a plurality of neural networks 22, such as calculating a weighted average of the output data from the plurality of neural networks 22, and the like.

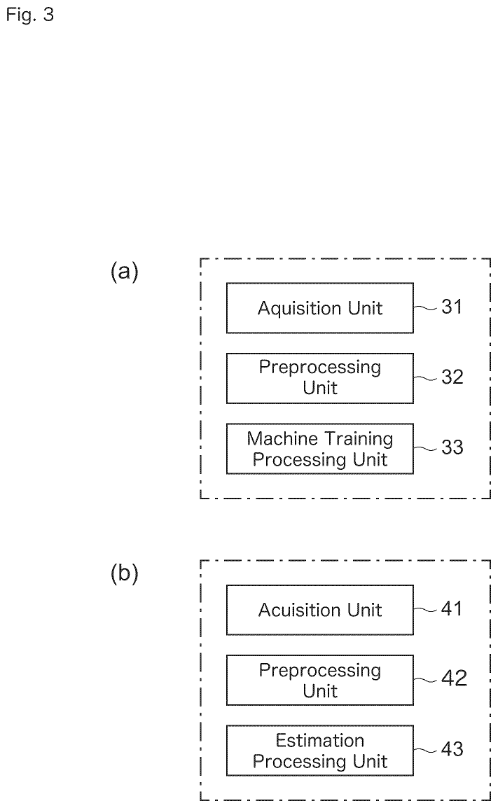

Further, as exemplified in FIG. 3A, the control unit 11 of the visual field estimation apparatus 1 executing the machine learning process, functionally comprises an acquisition unit 31, a preprocessing unit 32, and a machine learning processing unit 33.

Hereinbelow, first, an example where the machine learning model 20 is provided with the following neural networks 22 is to be explained:
neural network 22a receiving three-dimensional structure information of the left eye retina as input data, and outputting central 24-degree visual field change information of the left eye;
neural network 22b receiving image information of the left eye retina as input data, and outputting central 24-degree visual field change information of the left eye;
neural network 22c receiving three-dimensional structure information of the right eye retina as input data, and outputting central 24-degree visual field change information of the right eye;
neural network 22d receiving image information of the right eye retina as input data, and outputting central 24-degree visual field change information of the right eye;
neural network 22e receiving three-dimensional structure information of the left eye retina as input data, and outputting central 10-degree visual field change information of the left eye;
neural network 22f receiving image information of the left eye retina as input data, and outputting central 10-degree visual field change information of the left eye;
. . .
(in this case, eight neural networks in total, covering the difference of input data, difference of the left eye and the right eye, and the difference in the type of output data).

Here, for the neural network 22 receiving three-dimensional structure information as input data, a neural network suitable for machine learning of three-dimensional information is selected. An example of such a neural network 22 is a three-dimensional CNN (Convolutional Neural Network). Of course, this is only an example, and the neural network does not have to be a CNN.

Further, for the three-dimensional CNN, a widely known CNN, such as EfficientNet3D (https://github.com/shijianjian/EfficientNet-PyTorch-3D), ResNet3D, etc., can be adopted, and thus, detailed explanation therefor is omitted here.

Whereas, for the neural network 22 receiving image information as input data, a neural network suitable for machine learning of two-dimensional information is selected. An example of such a neural network 22 is a three-dimensional CNN. Of course, this is only an example, and the neural network does not have to be a CNN. In this example, for the two-dimensional CNN, a widely known CNN, such as EfficientNet, ResNet, etc., can be adopted, and thus, detailed explanation therefor is omitted here.

When the machine learning model 20 of this example is subjected to machine learning, a user of the visual field estimation apparatus 1 obtains, in advance, regarding a plurality of eyes to be examined of a plurality of patients, three-dimensional structure information of the retina and two-dimensional image information of the retina at each of a plurality of time points of examination, as well as visual field related information of each eye to be examined at each time point of examination. The visual field related information may be a temporal change of the threshold value itself at each measurement point included in the visual field, a temporal change of a visual field index such as a MD value, or a temporal change of information obtained on the basis of the visual field index or the threshold value such as a total deviation, a pattern deviation, etc. (For example, in case of the MD value, the temporal change thereof is a MD slope. In the explanation below, the MD value is used as a main example, but the present aspect is not limited thereto. In the explanation below, when “visual field index” is used, the expression also includes information obtained on the basis of the visual field index and the threshold value, such as a total deviation, a pattern deviation, and the like.) The visual field related information at the corresponding time point of examination, is obtained on the basis of actual measurement values of the threshold values of the visual field range (the visual field range corresponding to the output data from the neural network 22, such as the central 24-degree, the central 10-degree, etc.) obtained at a plurality of time points in the past, regarding the eye to be examined. For such preparation by the user, a procedure same as the conventional examination procedure can be adopted.

For example, as mentioned above, the visual field related information at the corresponding examination time point shows a temporal change of a visual field index or a threshold value. This can be obtained by a regression line y=at+b (wherein, t represents time (time and date), a represents a slope, and b represents an intercept) regarding visual field indices and threshold values at the respective measurement points obtained at a plurality of times of visual field examination relative to the time axis. As described below, in the present aspect, parameters subjected to statistical processing, such as this regression line, are used as training information, and thereby, machine learning capable of reducing the influences of the measurement variation of the visual field can be realized.

Then, a user of the visual field estimation apparatus 1 stores entries in the storage unit 12 regarding each eye to be examined and each examination time point, each entry being composed of eye identification information unique to each eye to be examined, information specifying whether the eye to be examined is the right eye or the left eye, examination time point information representing the examination time point, image information or three-dimensional structure information of the retina at the examination time point (hereinbelow, referred to as information to be input) and, visual field change information of the corresponding eye to be examined, in association with each other.

The user of the visual field estimation apparatus 1 provides an instruction to start the machine learning processing by operating the visual field estimation apparatus 1. The acquisition unit 31 of the visual field estimation apparatus 1 acquires the entries stored in the storage unit 12 by reading out the entries, for example, one by one in a predetermined order (in the order of acquisition or at random). Also, a general machine learning processing technology, such as batch processing, can be used here. In that case, the acquisition unit 31 reads out entries of the number corresponding to the size of a mini-batch, and subjects the read-out entries to the machine learning processing.

The preprocessing unit 32 executes a predetermined preprocessing to the information to be input included in the entries acquired by the acquisition unit 31, and outputs the resultant to the machine learning processing unit 33. For example, the preprocessing unit 32 executes a widely known process such as a predetermined noise reduction process, noise addition process, histogram equalization process, etc., to the three-dimensional structure information which is the information to be input. Further, the preprocessing unit 32 may execute a process such as reducing/magnifying, inversion/rotation relative to a predetermined face or axis, etc., within the three-dimensional space (two-dimensional space, if the information to be input is two-dimensional information) of the information to be input.

The machine learning processing unit 33 outputs the preprocessed image information of the retina of the eye to be examined that has been output from the preprocessing unit 32, to the input unit 21 of the machine learning model 20 as input data, and also, further outputs the information specifying whether the eye to be examined is the right eye or the left eye, and information representing that the input data is the image information of the retina, to the input unit 21.

Further, the machine learning processing unit 33 outputs the preprocessed three-dimensional structure information of the retina of the eye to be examined that has been output from the preprocessing unit 32, to the input unit 21 of the machine learning model 20 as input data, and also, further outputs the information specifying whether the eye to be examined is the right eye or the left eye, and information representing that the input data is the three-dimensional structure information of the retina, to the input unit 21.

Here, if the eye to be examined is “the left eye”, the input unit 21 of the machine learning model 20 outputs the image information of the retina to the neural networks 22b and 22f which correspond to the left eye, as input data.

Also, the input unit 21 outputs the three-dimensional structure information of the retina to the neural networks 22a and 22e which correspond to the left eye, as input data.

The output unit 23 of the machine learning model 20 synthesizes the output data from the neural network 22a and the output data from the neural network 22b, which are in the same output data type (same corresponding visual field range), and outputs the synthesized data as an estimation result of the central 24-degree visual field change information.

Similarly, the output unit 23 synthesizes the output data from the neural network 22e and the output data from the neural network 22f, and outputs the synthesized data as an estimation result of the central 10-degree visual field change information.

This synthesis by the output unit 23 is performed, for example, by generating a weighted average between the estimation value of the visual field change information included in the output data from the neural network 22a, and the estimation value of the visual field change information included in the output data from the neural network 22b, and using the generated weighted average as the visual field change information to be output, and the like. Here, the weight applied to each output data, as well as the parameters for the neural networks 22a, 22b, can be recursively updated by the machine learning processing unit 33.

The machine learning processing unit 33 receives the output data output from the output unit 23 of the machine learning model 20, compares the output data with the visual field change information of the corresponding visual field range included in the entry acquired by the acquisition unit 21, and based on the difference therebetween, updates the weight used by the output unit 23 of the machine learning model 20 and the parameter of the neural network 22 which outputs the output data of the corresponding visual field range. For the method for updating the parameter, etc., widely known methods such backpropagation, etc., can be adopted.

Specifically, on the basis of the difference between the central 24-degree output data obtained by synthesizing the output data from the neural network 22a and the output data from the neural network 22b, and the visual field change information of the corresponding visual field range, i.e., the central 24-degree, included in the entry acquired by the acquisition unit 21, the machine learning processing unit 33 updates the weight used when the output unit 23 of the machine learning model 20 synthesizes the output data from the neural network 22a and the output data from the neural network 22b, and the parameters of the neural network 22a and the neural network 22b.

Further, on the basis of the difference between the central 10-degree output data obtained by synthesizing the output data from the neural network 22e and the output data from the neural network 22f, and the visual field change information of the corresponding visual field range, i.e., the central 10-degree, included in the entry acquired by the acquisition unit 21, the machine learning processing unit 33 updates the weight used when the output unit 23 of the machine learning model 20 synthesizes the output data from the neural network 22e and the output data from the neural network 22f, and the parameters of the neural network 22e and the neural network 22f.

Similarly, regarding the right eye, the visual field estimation apparatus 1 performs machine learning of the weight to be used for the neural networks 22c, 22d, the neural networks 22g, 22h, and the syntheses of the output data from these.

The control unit 11 of the visual field estimation apparatus 1 can set the machine learning model 20 to a state that the relationship between the input and the output has been machine-learned, the input being the three-dimensional structure information or the image information of the retina, and the output being the visual field change information. In addition, the control unit 11 of the visual field estimation apparatus 1 can set the machine learning model 20 to a state that the relationship between the input and the information to be output has been machine-learned, the input being the three-dimensional structure information or the image information of the retina, and the information to be output being not only the visual field change information, but also the visual field related information such as the threshold value itself at each measurement point, a visual field index such as the MD value (including information obtained on the basis of the visual field index or the threshold value, such as the total deviation, the pattern deviation, etc.)

Also in this case, with respect to the visual field related information to be used in machine learning (the threshold value at each measurement point, the visual field index (including information obtained based on the visual field index or the threshold value, such as the total deviation, the pattern deviation, etc.), and the like), a regression line y=at+b (wherein, t represents time (time and date), a represents a slope, and b represents an intercept) is obtained regarding the temporal change of the threshold value, the visual field index obtained therefrom, etc., relative to the time axis, on the basis of the actual measurement values of the threshold value of a predetermined visual field range of the eye to be examined obtained at a plurality of time points in the past. Time t of the examination time point at which the three-dimensional structure information or the image information of the retina, i.e., input data, is obtained, is assigned to the regression line equation as above, and the solution of the equation becomes the visual field related information (threshold value, visual field index, etc.) at the corresponding examination time point. In case of the threshold value, a regression line is obtained at each measurement point, and time t of the examination time point is assigned to the corresponding regression line equation to obtain a solution value at each measurement point. Also in this case, parameters subjected to statistical processing, such as this regression line, are used as training information, and thereby, machine learning capable of reducing the influences of the measurement variation of the visual field can be realized.

### [Using Machine Learning Model]

Using the machine learning model 20 which has been machine-trained as above, the visual field estimation apparatus 1 estimates the visual field change information of a patient (person undergoing an eye examination) as below.

As exemplified in FIG. 3B, the control unit 11 of the visual field change information executing an estimation process functionally comprises an acquisition unit 41, a preprocessing unit 42, estimation processing unit 43.

A user of the visual field estimation apparatus 1 sequentially sets the right and left eyes of a patient to the eye to be examined, and acquires information such as (two-dimensional) image information of the retina of the eye to be examined, three-dimensional structure information of the retina of the eye to be examined, etc., which is the same type of information as the information used in the machine learning process of the machine learning model 20, by using OCT, etc.

Then, the user operates the visual field estimation apparatus 1 to give instructions to start an estimation process. The control unit 11 of the visual field estimation apparatus 1 receives the input of the (two-dimensional) image information of the retina of the eye to be examined, or the three-dimensional structure information of the retina of the eye to be examined, acquired by the user through the acquisition unit 41, and also receives information indicating whether the relevant eye to be examined is the right eye or the left eye.

The preprocessing unit 42 applies a predetermined preprocessing to at least one of the image information and the three-dimensional structure information acquired by the acquisition unit 41. This preprocessing applied by the preprocessing unit 42 is the same as the preprocessing by the preprocessing unit 32 applied to the information to be input in the machine learning processing, and thus, the explanation therefor is not repeated here.

From among the information output from the preprocessing unit 42, the estimation processing unit 43 outputs a preprocessed result of the image information of the retina of the eye to be examined to the input unit 21 of the machine learning model 20, as input data, and further outputs, to the input unit 21, information specifying whether the eye to be examined is the right eye or the left eye, and information indicating that the input data is the image information of the retina.

Further, from among the information output from the preprocessing unit 42, the estimation processing unit 43 outputs a preprocessed result of the three-dimensional structure information of the retina of the eye to be examined to the input unit 21 of the machine learning model 20, as input data, and further outputs, to the input unit 21, information specifying whether the eye to be examined is the right eye or the left eye, and information indicating that the input data is the three-dimensional structure information of the retina.

The machine learning model 20 outputs each input data to the corresponding neural network 22, and outputs the output data (in case of using output data from a plurality of neural networks 22, synthesis data of the output data) output from the relevant neural network 22.

Here, there are two types of output data, namely, the visual field change information with regard to the central 24-degree visual field of the eye to be examined, and the visual field change information with regard to the 10-degree visual field of the eye to be examined.

The estimation processing unit 43 uses the output data from the machine learning model 20, and outputs the relevant output data itself, or information obtained by subjecting the output data to a predetermined process, to the display unit 14. Here, the predetermined process is, for example, a process of estimating a future visual field of the patient. An example of applying this process will be explained below, with reference to an example of the output.

### [Display Example of Output]

Here, an example of the information display using the output data obtained by the estimation processing unit 43 is to be explained. According to the above example of the present aspect, the visual field change information relating to the central 24-degree visual field or the central 10-degree visual field is obtained with regard to each eye of the patient whose visual field index has been obtained, and thus, the numeral value of each information can be output as it is.

Also, instead of the visual field change information itself estimated by the machine learning model 20, the visual field estimation apparatus 1 may generate an estimation result of a current or future visual field by using the current visual field information of the patient (this can be actually measured, or can be estimated by using a neural network, etc., in which the relationship between the output from the OCT, etc., and the visual field has been machine-learned) and the visual field change information estimated by the machine learning model 20, and output the generated result.

In this example, the visual field estimation apparatus 1 uses the information of the current visual field with regard to the patient's eye of which the visual field change information has been obtained to generate information of the visual field at a predetermined time point in the future, after the change represented by the visual field change information, with regard to the patient's eye. For example, in case that the visual field change information is a MD slope, and the visual field change information of the central 24-degree visual field is estimated as −1.1 dB/year, the MD value at each measurement point within the central 24-degree visual field annually becomes approximately 0.88 times of the MD value of the previous year, in average. Therefore, in this example, the visual field estimation apparatus 1 estimates the central 24-degree visual field of five years later by calculating T′=T×0.88⁵, wherein T represents a current MD value at a measurement point within the central 24-degree visual field, and T′ represents a MD value five years later at the measurement point. This means that, when the Y-axis shows a threshold value of the visual field, and the X-axis shows time, a linear line for estimating a time-series change of the visual field of the patient's eye to be examined can be obtained by changing the position of the intercept on the Y-axis without changing the slope of the regression line

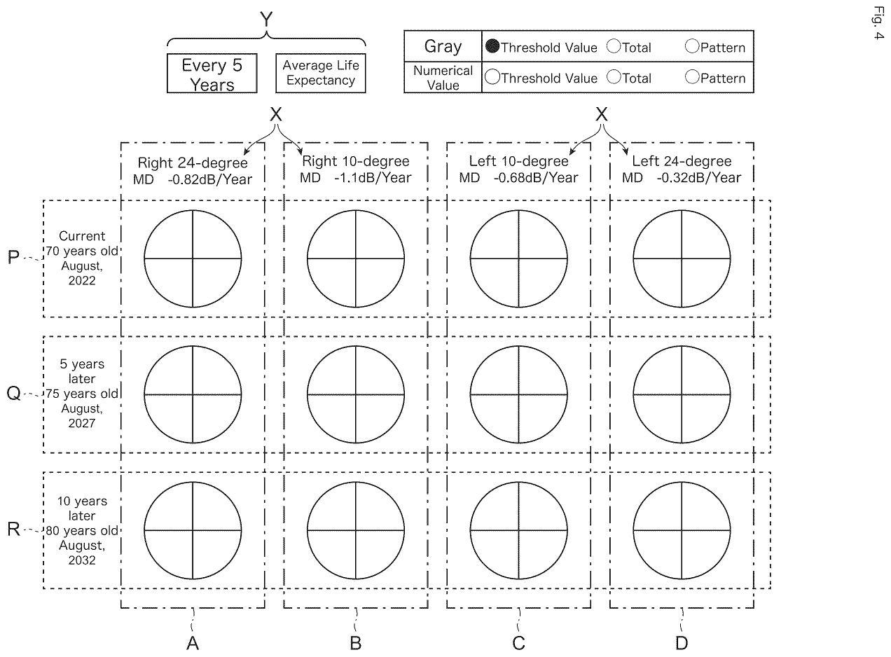

As the above example, the visual field estimation apparatus 1 estimates the MD value at a predetermined time point in the future. Further, the visual field estimation apparatus 1 can use a machine learning model 20 in which the output from the OCT, etc., and the visual field change information with regard to a threshold value at each measurement point have been machine learned, and obtain an estimation value of the threshold value at each measurement point, in the same way as the above example. In this example, the visual field estimation apparatus 1 displays a screen as exemplified in FIG. 4, and outputs an estimation result of the visual field at each predetermined time period, or at least one time point in the future determined on the basis of the average life expectancy of the patient.

The screen example shown in FIG. 4 shows an example displaying an estimation result of a patient's visual field by an image of a grayscale visual field, which is widely known as an output from the Humphrey Field Analyzer. For the sake of convenience, FIG. 4 shows an image of the grayscale visual field by a simple circle, but actually, a gray-colored image is shown in a part corresponding to the visual field measurement point within the circle.

In this example, the visual field estimation apparatus 1 displays visual fields estimated with regard to both of the central 24-degree visual field and the central 10-degree visual field of the patient's right/left eye, respectively, at a predetermined time point, from the left in the column direction, i.e., (A) the right eye central 24-degree, (B) the right eye central 10-degree, (C) the left eye central 10-degree, and (D) the left eye central 24-degree, in this order. Further, each visual field information may be displayed in association with the visual field change information. For example, regarding the right eye central 24-degree, if the estimated visual field change information is −0.82 dB, the visual field estimation apparatus 1 may display the characters “−0.82 dB” in association with column A (X). The displayed value can be not only the value of the MD slope (the visual field change information relating to the MD value), but also a VFI, or a TD value. Further, the displayed values may be switchable.

According to this example, the visual field estimation apparatus 1 displays the above-mentioned four types of grayscale visual field images by arranging them, in time-series order, in the row direction, at each of a plurality of time points, which are (P) current, (Q) five years later, and (R) ten years later. As a matter of course, this is only an example, and the visual field estimation apparatus 1 may display images in different ways For example, the above-mentioned plurality of time points may not be every five years, but may be determined in view of the average life expectancy, or a switching button (Y) may be arranged so that the user can perform switching therebetween as he/she desires. In addition, the visual field estimation apparatus 1 may display visual field estimation results at a larger number of time points. In this case, the number of rows (or columns) which can be displayed is limited due to the size of the screen, a known interface such as a scroll bar may be used so to enable the display by scrolling. Alternatively, the display may be enabled by switching.

Further, in case that the time in view of the average life expectancy is applied, information regarding the age and sex of the patient is required. However, if the visual field estimation apparatus 1 has not received such information, the switching button (Y) may be fixed at times other than the times taking the average life expectancy into account, such as every five years, and displayed in a way showing that the switching operation is disabled (for example, shown in gray out).

When the visual field estimation apparatus 1 has received the age and sex information, the visual field estimation apparatus 1 acquires the average life expectancy of the patient using the average life expectancy table (may be the table published by Ministry of Health, Labour and Welfare, etc.). Specifically, in case that the patient is a male of 65 years old (current), the average life expectancy is obtained as 20 years.

The visual field estimation apparatus 1 sets the time point corresponding to the first row as current, sets the time point corresponding to the last row as 20 years later, and equally divides the portion between the first and last rows into the number of rows to be displayed to determine time points corresponding to the intermediate rows. For example, in case that the three rows are displayed, the visual field estimation apparatus 1 estimates the visual field information of current, 10 years later, and 20 years later.

Further, in the above example, when the average life expectancy is not taken into account, the time points are set in every five years, but this is only an example. In accordance with the instructions or operations of the user, the visual field estimation apparatus 1 can change the time points as desired, such as every three years, every two years, and the like, and update the display by obtaining the visual field estimation result at each time that has been set. Taking also into account such a case, the visual field estimation apparatus 1 may also show the visual field estimation of how many years later is referred to by the image (P, Q, R . . . ) of each row. For example, the visual field estimation apparatus 1 may show a result obtained by adding the current date/time and the date/time until the estimated time point in the future such as “five years later, March 2027” in association with the image on a specific row. Further, in case that the age and the date of birth of the patient is known, the patient's age at the specific time point such as “five years later, 70 years old, March 2027” may also be shown.

In addition, in the above, the visual field information is shown by a grayscale visual field image, but this also is only an example. The visual field estimation apparatus 1 may show a numeral value for the estimation value of the threshold value at each measurement point, similar to the Humphrey Field Analyzer, or may show the visual field information by using the total deviation, or by using the pattern deviation. Further, in case that the total deviation or the pattern deviation is used, the value at each measurement point may be shown by a numerical value, or may be shown by a grayscale pattern. In this case, the visual field estimation apparatus 1 separately prepares a machine learning model 20 which has been directly machine-trained on the relationship between OCT outputs, etc., and values obtained based on visual field indices or threshold values, such as total deviation or pattern deviation etc. The visual field estimation apparatus 1 may directly obtain the value obtained based on the visual field index or the threshold value, such as the total deviation, the pattern deviation, etc. using the machine learning model 20 and may show the obtained value using a numerical value or a grayscale visual field image.

Namely, there are at least six ways of display in total, which are:
three ways for the grayscale visual field, i.e., the threshold value, the total deviation, and the pattern deviation; and,
three ways for the numerical value, i.e., the threshold value, the total deviation, and the pattern deviation.

The visual field estimation apparatus 1 may show a selection button for the user to select a way of display and display the information in the selected way. At this time, the visual field estimation apparatus 1 may show information indicating which way is selected for displaying the image, on the screen.

Further, the visual field estimation apparatus 1 may output the displayed screen image to a printer to print the image, in accordance with instructions by the user.

### [Process for Handling the Left Eye and the Right Eye in Common]

In the above example, the relationship of the three-dimensional structure information, etc., of the retina of the left eye and the retina of the right eye, respectively, with the visual field change information is separately machine-learned by the different neural networks 22. However, if the information of the left eye and the information of the right eye can be commonly machine-learned by the same neural network 22, the amount of training information for machine learning becomes two-times, resulting in efficient machine learning. Regarding the visual field information of the right/left eyes, except that the left and right are inverted, there are no reasonable reasons that the visual field change information of the respective visual field information are different. Therefore, according to the present aspect, the visual field estimation apparatus 1 may execute the machine learning process and the estimation process as follows.

In this example, the left eye or the right eye is previously determined as a reference. Hereinbelow, the left eye is determined as a reference, and the neural networks 22 provided in the machine learning model 20 are, for example:
neural network 22a receiving the three-dimensional structure information of the left eye retina as input data, and outputting the central 24-degree visual field change information of the left eye,
neural network 22b receiving the image information of the left eye retina as input data, and outputting the central 24-degree visual field change information of the left eye,
neural network 22e receiving the three-dimensional structure information of the left eye retina as input data, and outputting the central 10-degree visual field change information of the left eye,
neural network 22f receiving the image information of the left eye retina as input data, and outputting the central 10-degree visual field change information of the left eye 22f,
(four neural networks in total each being different in the input data, and in the type of the output data).

Similar to the above-mentioned case that the machine learning is performed separately to the right eye and to the left eye, the user performing the machine learning process of this machine learning model 20 stores entries in the storage unit 1, with respect to each eye to be examined and each examination time point, each entry having the eye identification information unique to the eye to be examined, the information specifying whether eye to be examined is the right eye or the left eye, the examination time point information representing the examination time point, the image information or the three-dimensional structure information of the retina at the examination time point (information to be input), and the visual field change information of the corresponding eye to be examined, associated with each other.

When the visual field estimation apparatus 1 starts the machine learning process, the acquisition unit 31 sequentially reads out and acquires the entries stored in the storage unit 12 one by one, and the preprocessing unit 32 applies a predetermined preprocessing to the information to be input included in the entry acquired by the acquisition unit 31, and outputs the preprocessed information to the machine learning processing unit 33.

Then, the machine learning processing unit 33 examines whether the eye to be examined relating to the acquired entry is the left eye or the right eye, and if the eye to be examined is not the left eye, i.e., the reference eye, but the right eye, the machine learning processing unit 33 inverts the right and the left in the preprocessed information to be input. Also, if the acquired entry includes threshold value information of the visual field, the machine learning processing unit 33 inverts the right and the left of the threshold value of the relevant visual field. Here, the right/left inversion refers to mirror-symmetrical inversion of the image information or the three-dimensional structure information of the retina of the eye to be examined, and the measurement point (and the threshold value corresponding thereto) in the visual field of the eye to be examined, relative to the face parallel to the sagittal plane of the human body.

If the eye to be examined relating to the acquired entry is the left eye, i.e., the reference eye, the machine learning processing unit 33 does not execute the right/left inversion.

Then, the machine learning processing unit 33 outputs the image information of the retina preprocessed by the preprocessing unit 32 (in case of the image information relating to the right eye, right/left inverted image information of the retina), to the input unit 21 of the machine learning model 20 as input data thereto, and further outputs information representing that the input data is the image information of the retina, to the input unit 21.

Also, the machine learning processing unit 33 outputs the preprocessed three-dimensional structure information of the retina of the eye to be examined output from the preprocessing unit 32 (in case of the three-dimensional structure information relating to the right eye, right/left inverted three-dimensional structure information of the retina), and further outputs information representing that the input data is the three-dimensional structure information of the retina, to the input unit 21.

The input unit 21 of the machine learning model 20 outputs the input data, i.e., the image information of the retina (regardless of whether the eye to be examined is the left eye or the right eye) to the neural networks 22b, 22f.

Also, the input unit 21 outputs the input data, i.e., the three-dimensional structure information of the retina (regardless of whether the eye to be examined is the left eye or the right eye) to the neural networks 22a, 22e.

The output unit 23 of the machine learning model 20 synthesizes the output data from the neural network 22a and the output data from the neural network 22b, both having the same output data type (corresponding visual field range), and outputs the synthesized data as an estimation result of the central 24-degree visual field change information.

Similarly, the output unit 23 also synthesizes the output data from the neural network 22e and the output data from the neural network 22f, and outputs the synthesized data as an estimation result of the central 10-degree visual field change information. Here, the synthesis by the output unit 23 is the same as the synthesis in the already explained example, and thus, the repeated explanation is omitted here.

The machine learning processing unit 33 receives the output data output from the output unit 23 of the machine learning model 20, compares the received output data with the visual field change information of the corresponding visual field range include in the entry acquired by the acquisition unit 21, and, on the basis of the difference found by the comparison, updates the weight to be used by the output unit 23 included in the machine learning model 20, and the parameter of the neural network 22 which outputs the output data of the corresponding visual field range. For the method for updating the parameter, etc., a widely known method such as backpropagation, etc., can be adopted.

Further, the visual field change information estimation process of the patient using the machine learning model that has been machine-trained as in the example, is performed as follows. In this example, while the right and left eyes of the patient to be estimated is sequentially set as the eye to be examined, the user of the visual field estimation apparatus 1 acquires information of the same type as the information used in the machine learning process of the machine learning model 20, such as the (two-dimensional) image information of the retina or the three-dimensional structure information of the retina of the eye to be examined, etc., using the OCT, and the like.

Then, the user operates the visual field estimation apparatus 1, and instructs to start the estimation process. The control unit 11 of the visual field estimation apparatus 1 receives the input of the (two-dimensional) image information of the retina of the eye to be examined and the three-dimensional structure information of the retina of the eye to be examined, which have been acquired by the user through the operation of the acquisition unit 41. The control unit 11 also receives the information indicating whether the eye to be examined is the right eye or the left eye.

Also in this example, the preprocessing unit 42 applies predetermined preprocessing to the image information or the three-dimensional structure information that have been input. The processing by this preprocessing unit 42 is also the same as the operation of the preprocessing unit 32 applied to the information to be input in the machine learning processing, and thus, the repeated explanation therefor is omitted here.

The estimation processing unit 43 determines whether the eye to be examined corresponding to the image information, etc., that has been input, is the right eye or the left eye. The determination may be done through the input of information by the user.

When the estimation processing unit 43 determines that the eye to be examined corresponding to the input image information, etc., is not the left eye, i.e., the reference eye, but the right eye, the right/left inversion is performed to both of the image information and the three-dimensional structure information of the retina of the eye to be examined, which have been output from the preprocessing unit 42.

Then, from among the output from the preprocessing unit 42, the preprocessed result of the retina image information of the eye to be examined (when the eye to be examined is the right eye, the image information after the right/left inversion) is output to the input unit 21 of the machine learning model 20 as input data, and information indicating that the input data is the retina image information is further output to the input unit 21.

Also, from among the output from the preprocessing unit 42, the estimation processing unit 43 outputs the preprocessed result of the three-dimensional structure information of the retina of the eye to be examined (when the eye to be examined is the right eye, the three-dimensional structure information after the right/left inversion) is output to the input unit 21 of the machine learning model 20 as input data, and information indicating that the input data is the three-dimensional structure information of the retina is further output to the input unit 21.

The machine learning model 20 outputs each of the input data to the corresponding neural network 22. Here, the input data, that is, the retina image information (regardless of whether the eye to be examined is the left eye or the right eye) is output to the neural network 22b, 22f. Further, the machine learning model 20 outputs the input data, that is, the three-dimensional structure information of the retina (regardless of whether the eye to be examined is the left eye or the right eye) to the neural networks 22a, 22e.

Then, the output unit 23 of the machine learning model 20 synthesizes the output data from the neural network 22a and the output data from the neural network 22b, both being the same type of output data (having the same corresponding visual field range), and outputs the synthesized data as the estimation result of the central 24-degree visual field change information.

Similarly, the output unit 23 synthesizes the output data from the neural network 22e, and the output data from the neural network 22f, and outputs the synthesized data as the estimation result of the central 10-degree visual field change information.

The estimation processing unit 43 uses the output data from the machine learning model 20, and outputs the output data as it is, or the information obtained by using the output data, to the display unit 14.

### [Basic Operation Example]

The visual field estimation apparatus 1 of the present aspect basically has the above-mentioned configuration, and operates as in the example below. The visual field estimation apparatus 1 of the present aspect performs operations for executing the machine learning processing, and operations for executing the estimation using the machine learning model 20 that has been machine-trained, respectively.

The user executing the machine learning processing of the machine learning model 20 collects, from the past examination results of a plurality of patients, at least one of the three-dimensional structure information of the retina by the OCT and the retina image information (information to be input), as well as the visual field related information which is the temporal change of the visual field index or the threshold value on the basis of the visual field information of a predetermined range (range of central 30-degree, central 24-degree, central 10-degree, or a mixture of central 24-degree and central 10-degree) obtained on a plurality of days until the acquisition date of the information to be input. Here, the visual field information may be the one actually measured by the Humphrey Field Analyzer, etc., and the visual field related information which is the temporal change can be obtained as visual field indices such as MD values, etc., of a plurality of days, or parameters of regression lines for the threshold values.

Regarding the same eye to be examined of the same patient, if there are sets of the information to be input and the visual field information acquired on the substantially different acquisition dates, the user executing the machine learning processing can obtain the visual field change information such as a MD slope, on the basis of the visual field information included to each set, by, for example, a conventional method.

The user executing the machine learning processing issues the eye identification information unique to every eye to be examined, and stores a plurality of entries in the storage unit 12, each entry including, in association with each other, the eye identification information unique to the eye to be examined, examination time/date information indicating the time/date of the examination time point (can be the date when the three-dimensional structure information, etc., is acquired), the three-dimensional structure information, etc., of the retina at the examination time point (information to be input), the visual field information at the relevant time point, and the obtained visual field change information.

Then, the user executing the machine learning processing operates the visual field estimation apparatus 1, and instructs to start the machine learning processing. The visual field estimation apparatus 1 sequentially reads out the entries stored in the storage unit 12, one by one. Then, the visual field estimation apparatus 1 applies preprocessing, for example, a predetermined noise reduction processing, to the information to be input (for example, three-dimensional structure information) included in the entry.

Here, the visual field estimation apparatus 1 outputs the preprocessed information to be input to the machine learning model 20, as input data. The machine learning model 20 outputs output data corresponding to the input data.

According to an example of the present aspect, as already mentioned above, the machine learning model 20 has been machine-trained so as to output visual field change information (MD slope, etc.) at a predetermined visual field range (for example, central 24-degree, central 10-degree, or a mixture of these). Then, the visual field estimation apparatus 1 receives the output data from this machine learning model 20, compares the output data with the visual field change information included in the read-out entry, and, on the basis of the difference found by the comparison, updates the parameter, etc., of the neural network included in the machine learning model 20 by backpropagation, and thereby, machine learning of the machine learning model 20 is executed.

According to an example of the present aspect, a plurality of machine learning models 20 may be obtained based on whether the eye to be examined is the left eye or the right eye, and based on which range of the visual field change corresponds to the visual field change information to be output, the machine learning models including:
machine learning model receiving the three-dimensional structure information of the left eye retina, etc., as the input data, and outputting the central 24-degree visual field change information with regard to the left eye;
machine learning model receiving the three-dimensional structure information of the right eye retina, etc., as the input data, and outputting the central 24-degree visual field change information with regard to the left eye;
machine learning model receiving the three-dimensional structure information of the left eye retina, etc., as the input data, and outputting the central 10-degree visual field change information with regard to the left eye;
machine learning model receiving the three-dimensional structure information of the right eye retina, etc., as the input data, and outputting the central 10-degree visual field change information with regard to the left eye;
Next, the operation of estimating the visual field change information of the patient by the visual field estimation apparatus 1, using the machine learning model 20 that has been machine-trained, will be explained.

The user who estimates the visual field change information of the patient acquires, by using the OCT, etc., information of the same type as the information used in the machine learning processing of the machine learning model 20, such as the three-dimensional structure information of the retina, with regard to the eye to be examined (the left eye and the right eye, respectively) of the patient to be estimated

The user inputs the acquired three-dimensional structure information of the retina to the visual field estimation apparatus 1. At this time, when the user inputs the three-dimensional structure information of the left eye retina of patient, the user instructs the visual field estimation apparatus 1 to use a machine learning model 20 which receives the three-dimensional structure information, etc., of the left eye retina as input data, and outputs the central 24-degree visual field change information of the left eye, and to use a machine learning model 20 which receives the three-dimensional structure information, etc., of the left eye retina as input data, and outputs the central 10-degree visual field change information of the left eye.

The visual field estimation apparatus 1 receives this information, applies preprocessing such as a predetermined noise reduction processing to the received information, and inputs the preprocessed information to the instructed machine learning models 20, respectively, and obtains estimation values for the visual field change information (MD slope, etc.) of the central 24-degree visual field, the visual field change information of the central 10-degree visual field, . . . , respectively output from the machine learning models 20.

Further, when the user inputs the three-dimensional structure information of the right eye retina of the patient, the user instructs the visual field estimation apparatus 1 to use a machine learning model 20 which receives the three-dimensional structure information, etc., of the right eye retina as input data and outputs the central 24-degree visual field change information of the right eye, and to use a machine learning model 20 which receives the three-dimensional structure information, etc., of the right eye retina and outputs the central 10-degree visual field change information of the right eye.

The visual field estimation apparatus 1 receives this information, applies preprocessing such as a predetermined noise reduction processing to the received information, and inputs the preprocessed information to the instructed machine learning models 20, respectively, and obtains estimation values for the visual field change information (MD slope, etc.) of the central 24-degree visual field, the visual field change information of the central 10-degree visual field, . . . , respectively output from the machine learning models 20.

By the above-mentioned processing, the visual field estimation apparatus 1 acquires visual field change information of the right eye central 24-degree, the right eye central 10-degree, the left eye central 10-degree, and the left eye central 24-degree, respectively. Further, the visual field estimation apparatus 1 receives the current visual field information of the patient. This visual field information may be estimated by using, for example, a neural network, etc., that has been subjected to the machine learning processing to learn the relationship between the output from the OCT and the visual field (the central 10-degree and the central 24-degree of the right eye, and the central 10-degree and the central 24-degree of the left eye, respectively).

The visual field estimation apparatus 1 uses the estimated visual field information of the right eye central 24-degree of the patient, and the estimated visual field change information of the right eye central 24-degree visual field of the patient, to obtain estimated threshold values for the patient's visual field of 5 years later, and 10 years later. Similarly, the visual field estimation apparatus 1 obtains estimated threshold values for the patient's visual field of 5 years later, and 10 years later, with respect to the right eye central 10-degree, the left eye central 10-degree, and the left eye central 24-degree, respectively.

Then, as exemplified in FIG. 4, the visual field estimation apparatus 1 displays grayscale visual field images (P) based on the estimation values for (A) the right eye central 24-degree, (B) the right eye central 10-degree, (C) the left eye central 10-degree, and (D) the left eye central 24-degree, of the current; grayscale visual field images (Q) based on the estimation values for (A) the right eye central 24-degree, (B) the right eye central 10-degree, (C) the left eye central 10-degree, and (D) the left eye central 24-degree, estimated as values of five years later; and grayscale visual field images (R) based on the estimation values for (A) the right eye central 24-degree, (B) the right eye central 10-degree, (C) the left eye central 10-degree, and (D) the left eye central 24-degree, estimated as values of ten years later.

This example of display has already been explained, and thus, the explanation is not repeated here. According to the present aspect, the visual field change is estimated on the basis of the current three-dimensional structure information of the retina by the OCT and the current image information of the retina, and a future visual field is estimated on the basis of the estimated change, and the estimation result is displayed. Thereby, if the OCT examination, etc., is performed once, the future visual field can be estimated, and thus, the progression rate of the visual field defect can be judged at an early stage.

### [Example of Further Using Another Machine Learning Model]

In the explanation until here, the machine learning model 20 has been machine-trained so as to receive the three-dimensional structure information of the retina or the image information of the retina as input data, and output change information related to the visual field (visual field related information) such as a MD value of the corresponding eye to be examined, etc. Thereby, the visual field change information can be obtained using this machine learning model 20.

However, the machine learning model according to the present aspect is not limited to this example. The machine learning model used by the visual field estimation apparatus 1 according to the present aspect may be a machine learning model 20′ which receives, as input data, the output from the machine learning model 20, as well as information regarding other risk factors for glaucoma, such as intraocular pressure, corneal hysteresis, etc., and other information such as age, sex, etc., and generates output data including the visual field change information of the visual field.

Examples of the machine learning model 20′ includes; a multilayer perceptron having at least one hidden layer, Lasso regression model, Ridge regression model, Random Forest regression model, Support Vector regression model, and the like (the used kernel function may be any of linear, polynomial, and Gaussian), and one of these may be used by itself, but some of these may be combined as desired. The machine learning model 20′ is subjected to machine learning processing as below, using the machine learning model 20 explained above.

According to this example of the present aspect, first, the machine learning model 20 has been machine-trained by the machine learning processing which is already explained above. Also, another machine learning model is prepared as a machine learning model (hereinbelow, referred to as a visual field estimation model) which has been machine-trained so as to receive the three-dimensional structure information of the retina or the image information of the retina as input data, and to output visual field information of the eye to be examined at the time point when the three-dimensional structure information or the image information is obtained (a threshold value at each measurement point, etc.).

Next, each user who performs machine learning of the machine learning model 20′ acquires three-dimensional structure information and image information by the OCT, etc., at least once, and separately, performs measurements of the visual field at a plurality of time points, and collects information of a plurality of eyes to be examined (preferably, these eyes to be examined are different from the eyes to be examined used for the machine leaning of the machine learning model 20) of which actual measurement values of the visual field change information, such as MD slopes, etc. (referred to as actual measurement visual field change information) can be obtained.

Here, among the eyes to be examined, regarding an eye to be examined of which image information by the OCT, etc., was obtained only once or twice in the past, visual field change information which is the time-series information cannot be generated, and thus, information of such an eye to be examined is not used for the machine learning of the time-series information.

On the other hand, regarding an eye to be examined of which image information etc., by OCT, etc., was obtained for three times or more, at mutually different plurality of time points, the temporal change of the visual field index is obtained as follows. Here, information specifying the time point when each image information, etc., was obtained (examination time point) is recorded together with the image information, etc.

Namely, the user uses the visual field estimation apparatus 1 to sequentially input the input data to the machine learning model 20, each input data being the image information, etc., obtained regarding one eye to be examined at each of the plurality of time points. Then, the visual field estimation apparatus 1 obtains the output data output from the machine learning model 20, the output data being a plurality of pieces of visual field change information respectively corresponding to input data regarding each of the central 24-degree and the central 10-degree. Further, the visual field estimation apparatus 1 outputs the average value (can be an arithmetic average), the standard deviation, the number of samples, etc., of the visual field change information obtained here. Hereinbelow, for distinguishing, the average value of the visual field change information output here is referred to a “direct estimation value”.

Further, the visual field estimation apparatus 1 sequentially inputs the input data to the visual field estimation model, each input data being the image information, etc., regarding the eye to be examined that was input to the machine learning model 20. Then, the visual field estimation apparatus 1 arranges the visual field estimation results obtained by the visual field estimation model corresponding to the input image information, etc., on the time axis in accordance with the acquisition time point information of the corresponding image information, etc., obtains a regression line, etc., regarding values of the visual field index, such as MD value, etc., relative to the time axis, and obtains the visual field change information as a slope of the regression line (for distinguishing, referred to as “indirect estimation value”).

Further, when the number of pieces of the obtained visual field information (the number of samples) is larger than a predetermined number, the visual field estimation apparatus 1 can divide the time into a plurality of periods, obtain visual field change information for each sample obtained in each period, and output an average value (can be an arithmetic average) of the visual field change information corresponding to each period.

Also, at this time, with regard to the value of the visual field change information corresponding to each period, the visual field estimation apparatus 1 obtains a correlation coefficient such as Pearson's product-moment correlation coefficient, Spearman's rank correlation coefficient, etc., as well as a value relating to the p-value thereof. Here, the value relating to the p-value can be obtained by calculating a logarithm of an ordinary p-value regarding the correlation coefficient, and changing the sign (changing the sign to minus) thereof. In this case, in order to prevent divergence of the value, a value small enough to have no influence to the result, for example, 0.001, has been added to the p-value. Accordingly, the larger the value relating to the p-value, the higher the reliability.

When the value relating to the p-value exceeds a predetermined threshold, the reliability of the indirect estimation value can be determined as comparatively higher than the reliability of the direct estimation value. When the value relating to the p-value does not exceed the threshold, (for example, the value relating to the p-value is “0”), the reliability of the direct estimation value can be determined as higher than the reliability of the indirect estimation value. On the basis of the determination, either can be selectively used for the visual field change information of the eye to be examined.

Further, in another example, a weighted average of the direct estimation value and the indirect estimation value is obtained, and the obtained value can be used for the visual field change information of the eye to be examined. In this case, the value of the weight is subjected to machine learning, together with the machine learning model 20′.

Further, if the number of samples is 2 (or 2 or less), the p-value cannot be obtained. Therefore, regarding such an eye to be examined, the visual field estimation apparatus 1 sets the indirect estimation value as the average value of the indirect estimation values of the population to be learned or “0”, and sets the value relating to the p-value as “0”.

Further, with regard to the eye to be examined, the visual field estimation apparatus 1 obtains the average value obtained in the past, the standard deviation, the slope of the regression line thereof, and the p-value, etc., for the information regarding the risk factor of glaucoma, such as the intraocular pressure, etc. If the information of the intraocular pressure has not been obtained, the value is set to an average value (15 mmHg) of a normal person, or an average value of the population to be learned (mainly including a patient with onset of glaucoma). In this case, the slope of the regression line is “0”. This is merely an example, and if the information of the intraocular pressure has not been obtained, instead of the above-mentioned average value, the information of the intraocular pressure is treated as missing data, and a value estimated by a known method for estimating missing data (for example, MCflow: https://arxiv.org/pdf/2003.12628.pdf) can be set.

As risk factors for glaucoma, if not only the intraocular pressure, but also the CH value (corneal hysteresis) is obtained, the visual field estimation apparatus 1 can obtain an average value, etc., of the CH values. In case of the CH value, if there are no CH values obtained through the examination, the average value of the CH values of normal persons can be set as the average value of the CH value for the eye to be examined. Also for the CH value, if there are no CH values obtained through the examination, similar to the case of the information of the intraocular pressure mentioned above, the value can be treated as missing data, and a value estimated by using a known method for estimating the missing data can be set.

The visual field estimation apparatus 1 further acquires the age of the person having the eye to be examined, last time that the image information, etc., by the OCT., is obtained (age at the time of the last examination). At this time, information regarding the degree of nearsightedness, sex, information of the corneal thickness, etc., are acquired together.

By the above-mentioned processes, on the basis of the information regarding a plurality of eyes to be examined collected by the user, the visual field estimation apparatus 1 inputs the input data to the machine learning model 20′, with regard to each eye to be examined relating to the collected information, the input data including:
direct estimation value,
indirect estimation value, and
correlation coefficient, p-value, etc., regarding the indirect estimation value;
   and preferably, the input data further including at least one of the following:
average value, standard deviation, etc., of intraocular pressure,
average value, etc., of CH value,
age at the time of the last examination,
degree of nearsightedness,
sex,
corneal thickness.

On the other hand, the visual field estimation apparatus 1 compares the actual measurement visual field change information based on the actual visual field examination result of the eye to be examined with the output data from the machine learning model 20′, and adjusts each parameter of the machine learning model 20′ by a process such as backpropagation (in case of a multi-layered model), fitting (in case of a regression model), and the like, so that machine learning is performed regarding the relationship between each of the input data and the visual field change information.

The user who would like to estimate the visual field change information of the patient using this machine learning model 20′, acquires the three-dimensional structure information and the image information of the retina of the eye to be examined of the patient, by the OCT, etc., and inputs the acquired information to the visual field estimation apparatus 1. Further, the user inputs information such as an intraocular pressure examination result and a CH value examination result in the past of the patient's eye to be examined, the age the last time the image information, etc., was acquired (age at the time of the last examination), a degree of nearsightedness, sex, corneal thickness, and the like, to the visual field estimation apparatus 1, and instructs to estimate the visual field change information.

Using the machine learning model 20′ which has been machine-trained, the visual field estimation apparatus 1 obtains direct estimation values. Also, using a visual field estimation model which has been machine-trained to receive the three-dimensional structure information of the retina and the image information of the retina as input data, and to output the visual field information of the eye to be examined at the time point when the three-dimensional structure information and the image information are obtained, the visual field estimation apparatus 1 estimates a visual field, and obtains indirect estimation values as well as the correlation coefficient, p-value, etc., thereof, obtained based on the information of the estimated visual field.

The visual field estimation apparatus 1 obtains an average value, a standard deviation, a slope of a regression line thereof, a p-value, etc., regarding the past intraocular pressure examination results that have been input. Here, if there are no intraocular pressure examination results, the visual field estimation apparatus 1 sets the average value of the intraocular pressure to the average value (15 mmHg) of the normal person, or the average value of the population to be learned, or a value determined by using a predetermined missing data estimation method. In this case, the slope of the regression line is set to “0”.

Further, if the CH values (corneal hysteresis) are obtained, the visual field estimation apparatus 1 also obtains the average value, etc., thereof. Also regarding the CH value, if there are no CH values obtained by examinations, the visual field estimation apparatus 1 sets the average value of the CH values of the eye to be examined to the average value of the normal person or a value determined by using a predetermined missing data estimation method.

Then, the visual field estimation apparatus 1 inputs the input data to the machine learning model 20′, regarding the patient's eye to be examined, the input data including:
direct estimation value,
indirect estimation value,
correlation coefficient, p-value, etc., regarding the indirect estimation value,
average value, standard deviation, etc., of intraocular pressure;
   and preferably, the input data further including at least one of the following:
average value, etc., of CH value,
age at the time of the last examination,
degree of nearsightedness,
sex,
corneal thickness.

Then, the visual field estimation apparatus 1 obtains an estimation result of the visual field change information regarding the eye to be examined, that has been output from the machine learning model 20′. On the basis of the obtained estimation result, the visual field estimation apparatus 1 may output a visual field estimation result at a predetermined time point in the future. The display example of this has already been explained, and the repeated explanation thereof is omitted here.

Also in the case that the machine learning model 20′ is used, either the right eye or the left eye is set as a reference, and if the eye to be examined is not the reference eye, the right/left is inverted before the input regarding the data having right/left difference (three-dimensional structure information, image information, output from visual field estimation model, etc.), among the input data input to the machine learning model 20, and the machine learning model 20′.

Further, when the visual field estimation result based on the estimated visual field change information is output, the visual field estimation result is output using information indicating whether the estimation result relates to visual field change information of the right eye or the left eye.

Here, when the indirect estimation value is obtained, the visual field estimation model is used. However, the present aspect is not limited thereto. The indirect estimation value can be obtained on the basis of the actual examination result. In such a case, the correlation coefficient, the p-value, etc., regarding the indirect estimation value are also obtained based on the actual examination result.

Further, regarding the indirect estimation value, whether the estimation value obtained by the visual field estimation model is adopted or the estimation value obtained from the actual examination result is adopted, can be determined depending on the situation of the patient (or the person having the eye to be examined). For example, in the case of the eye to be examined of a person who has difficulty in vision fixation, the indirect estimation value obtained by the visual field estimation model can be selected. Accordingly, the visual field estimation apparatus 1 can determine to adopt which of the estimation value obtained by the visual field estimation model or the estimation value obtained from the actual examination result, by comparing the p-values of these and selecting the one having a larger p-value. In addition, a weighted average of the estimation value obtained by the visual field estimation model and the estimation value obtained by the actual examination result may be obtained by using a weight corresponding to each p-value, and the obtained result can be used as the indirect estimation value.

In the above, the estimation value for the change of the visual field index, such as MD value, etc. (visual field change information) is used for the direct estimation value or the indirect estimation value. However, the visual field estimation apparatus 1 can obtain at least one of the direct estimation value and the indirect estimation value, using the estimation value regarding the change of the visual field threshold value.

Namely, the visual field estimation apparatus 1 subjects the machine learning model 20 to machine learning processing so as to receive the input of the three-dimensional structure information and the image information of the retina obtained by the OCT, etc., and to output the value showing the change of the visual field threshold value at each measurement point, and thereby, obtains the value showing the change of the threshold value at each measurement point (direct estimation value). The threshold value at each measurement point is obtained at each of a plurality of time points by estimation, and using the threshold values at the plurality of time points at each measurement point, a regression line for each measurement point is obtained, and the value showing the change of the threshold value at each measurement point is obtained as a slope of the regression line, and the obtained value is used for the indirect estimation value.

### [Modification of Visual Field Estimation Result]

It is known that as the glaucoma progresses, and the visual field is partially deteriorated, deterioration of the visual field therearound progresses. In the above explanation, the visual field estimation is performed provided that the deterioration progresses at the same speed at all the measurement points in the entire visual field. However, in case of estimating the visual field after a long time (for example, after ten years or more), the partial deterioration should be taken into account.

According to an example of the present aspect, the above drawbacks can be solved by performing the visual field estimation using a variational auto-encoder (β-VAE) 50, together.

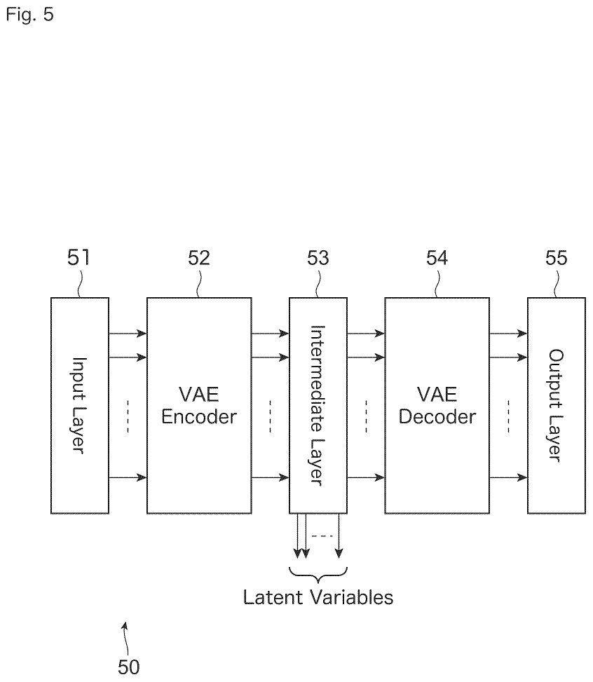

As exemplified in FIG. 5, the β-VAE 50 comprises an input layer 51, a VAE encoder 52, an intermediate layer 53, a VAE decoder 54, and an output layer 55. The intermediate layer 53 outputs values of latent variables output from the VAE encoder 52, and the number of latent variables included in the intermediate layer 53 (the number of nodes on the output side of the VAE encoder 52) is experimentally selected. For example, the number may be 4, 8, 16, 32, 64, etc.

Using a computer (here, referred to as a machine learning processing device) such as the visual field estimation apparatus 1, the user performs machine learning of the β-VAE 50 as below, the learning target thereof being a plurality of eyes to be examined of which the three-dimensional structure information and the image information of the retina by the OCT, etc., have been obtained, actual visual field examination results at a plurality of time points have been obtained, and a regression line of the visual field change has been obtained.

Using the visual field estimation apparatus 1, etc. (can be the machine learning device itself), the three-dimensional structure information and the image information of the retina obtained by OCT, etc., are input to the visual field estimation model that has been machine-trained, and the visual field at each time point of examination by OCT, etc., (threshold value at each measurement point in the visual field) is estimated. Here, threshold values are estimated at the measurement points (120 points in total) within the visual fields, i.e., both of the central 24-degree visual field (including 52 measurement points) and the central 10-degree visual field (including 68 measurement points). Of course this is only an example, and only the measurement points in the central 24-degree visual field, or only the measurement points in the central 10-degree visual field, can be used.

The machine learning processing device inputs the estimation result obtained here (estimation value of the threshold value at each of the 120 measurement points in total) as the input data to the input layer 51 of the β-VAE 50, and obtains the output from the output layer 55. Then, the machine learning processing device compares a value at each measurement point obtained based on the actual visual field examination result of the eye to be examined corresponding to the input data (a value at the measurement point, at a predetermined time point in the future, calculated by the regression line), with a value at each corresponding measurement point and output from the output layer 55, updates parameters such as a weight, bias between the layers included in the VAE encoder 52, VAE decoder 54, etc., of the β-VAE 50 using a method such as backpropagation, etc., to thereby perform the machine learning of the β-VAE 50.

When the visual field estimation apparatus 1 estimates the visual field information of the eye to be examined at a plurality of time points in the future, using the visual field change information estimated by the machine learning models 20, 20′, the β-VAE 50 that has been machine-trained as above is used to perform processes as follows. In this example, the visual field estimation apparatus 1 sequentially inputs the visual field information estimated for the eye to be examined at the above-mentioned plurality of time points (hereinbelow, referred to as the first estimated future visual field), to the β-VAE 50 that has been machine-trained. Then, the visual field estimation apparatus 1 acquires a value of a latent variable output from the intermediate layer 53 of the β-VAE 50 corresponding to the visual field information at each time point.

The visual field estimation apparatus 1 obtains a regression line regarding the plurality of points arranged with the Y-axis representing the value of each latent variable, and the X-axis representing the estimated future time point, obtains a correlation coefficient such as Pearson's product-moment correlation coefficient, Spearman's rank correlation coefficient, etc., regarding the mutually corresponding latent variable values at each time point, and obtains a p-value therefor (as already explained, a logarithm of the ordinary p-value is obtained, and the sign is changed to minus). At this time, the visual field estimation apparatus 1 can obtain and record the number of samples (the number of estimated future time points), together.

The visual field estimation apparatus 1 uses the regression line obtained regarding each latent variable, and estimates corresponding latent variable values at the time points after a long period of time (for example, time points after a period corresponding to the average life expectancy).

The visual field estimation apparatus 1 inputs the estimated latent variable value to each corresponding input node of the VAE decoder 54 of the β-VAE 50. Then, the visual field estimation apparatus 1 determines the estimation value of the threshold value at each measurement point in the visual field, output from the output layer 55, as the estimation value of the threshold value at each measurement point at the time point after a long period of time.

According to this example, at each measurement point within the visual field, prediction is performed taking into account the information regarding a measurement point around the relevant measurement point. Also, the visual field estimation apparatus 1 may further output an average value of the p-value of the latent variable, as an index for reliability.

In addition, with regard to the threshold values in the future (after a long period of time) obtained by the β-VAE 50 at each measurement point (in the above example, each of the 120 measurement points), and the values of the first estimated future visual field estimated regarding the measurement point at a plurality of time points, the visual field estimation apparatus 1 arranges the corresponding time point on the X-axis, and arranges the values on the Y-axis. The visual field estimation apparatus 1 obtains a regression line relating to the values of the first estimated future visual field estimated at the plurality of time points, by the least squares method, etc., the regression line passing through the points, on the above XY plane, corresponding to the threshold values in the future (after a long period of time) obtained by the β-VAE 50.

The slope of the regression line regarding each measurement point is the visual field change information of the relevant measurement point. With respect to each measurement point, the visual field estimation apparatus 1 can obtain a future estimated threshold value (referred to as second estimated future visual field), using the corresponding visual field change information, the past and current visual field (such as a threshold value of the visual field obtained by the actual examination, at each measurement point, a threshold value estimated by the visual field estimation model, etc.). The visual field estimation apparatus 1 can estimate the visual field after a long period of time as follows, instead of using the second estimated future visual field as it is.

On the basis of the p-value P1 relating the slope (visual field change information, without using the β-VAE 50) of the regression line used for obtaining the first estimated future visual field (referred to as the first regression line), and the average value P2av of the p-value P2 relating to the slope of the regression line obtained by using the β-VAE 50 for each measurement point (conveniently, referred to as the second regression line), the visual field estimation apparatus 1 obtains the weight relating to the first regression line as P1/(P1+P2av), and obtains the weight relating to the second regression line as P2av/(P1+P2av).

Then, using the slope value α1 of the first regression line at each measurement point and the slope value α2 of the second regression line at the relevant measurement point, the visual field estimation apparatus 1 obtains the slope a of the visual field change estimated relating to the relevant measurement point as: α=α1×P1/(P1+P2av)+α2×P2av/(P1+P2av).

Next, using the visual field information of the patient at a plurality of time points (which may be actually measured, or estimated by using a neural network, etc., in which the relationship between the output from the OCT, etc., and the visual field has been machine-learned), and the slope a of the visual field change obtained for each measurement point, the visual field estimation apparatus 1 obtains a regression line having a corresponding slope a for each measurement point, with respect to the relevant measurement point, by the least squares method for the threshold value information in the visual field at the plurality of time points.

Then, using the obtained regression line, a threshold value at each measurement point in the visual field after a predetermined long period of time is estimated, on the basis of the current visual field. The visual field estimation apparatus 1 outputs the obtained threshold values at each measurement point, by displaying the same as exemplified in FIG. 4.

In the above, the mixture ratio of the slope value α1 of the first regression line, and the slope value α2 of the second regression line relating to the relevant measurement point, are determined directly based on the respective p-values, but the present aspect is not limited thereto. The optimum value for the mixture ratio can be obtained experimentally by the machine learning of the mixture ratio machine learning model (may be a generally used multilayer perceptron, Lasso regression model, Ridge regression model, etc.). This mixture ratio machine learning model can be machine-trained using the estimated visual field in the future obtained by using the regression line (first regression line) for each measurement point at which the p-value (p-value before the logarithmic transformation and the sign inversion) is comparatively low (less than 0.05, etc.).

Further, the values to be mixed are not limited to the slope value α1 of the first regression line, and the slope value α2 of the second regression line relating to the relevant measurement point. The visual field estimation apparatus 1 can determine the mixture ratio of the slope values of the regression lines obtained from the actually measured visual fields, by using the above-mentioned mixture ratio machine learning model, calculate a weighted average of the first regression line slope value α1, the second regression line slope value α2 relating to the relevant measurement point, and the regression line slope obtained from the actually measured visual field, by using the obtained mixture ratio as the weight, and use the obtained weighted average value for the visual field estimation, etc., as a slope of the visual field change of the eye to be examined.

Using the obtained slope of the visual field change of the eye to be examined, the visual field estimation apparatus 1 estimates the visual field (threshold value at each measurement point in the visual field) of the sufficiently distant future (for example, after the time period corresponding to the average life expectancy). Then, the estimated threshold value of the visual field, and the threshold value actually measured at each measurement point in the visual field, are arranged, as points, on the XY plane with the Y-axis value representing the threshold value, and the X-axis value representing the time point of the measurement. A linear line is obtained in a way so that the line passes through the point on the XY plane corresponding to the threshold value of the estimated visual field of the sufficiently distant future, and the total sum of the distances from the relevant point to other points corresponding to the actually measured threshold values calculated by the least squares method becomes the minimum. The slope of this linear line represents the progression rate of the visual field (visual field change information) at the relevant measurement point.

Thereby, the visual field estimation apparatus 1 obtains the visual field change information at each measurement point, and on the basis of the obtained visual field change information, the status of the eye to be examined at a time point in the future can be estimated.

In the machine learning processing of the machine learning models 20, 20′, the input data can be subjected to a Data Augmentation process as mentioned below. For example, at the time of the machine learning of the machine learning model 20′, instead of the ordinary method of using the estimation value obtained by inputting the image data, etc., of the retina to the machine learning model 20, the machine learning processing of using the average value between the estimation value obtained by inputting the vertically inverted data to the machine learning model 20 and the estimation value obtained by inputting the (original) data before the inversion to the machine learning model 20, can be used.

1. A visual field estimation apparatus comprising:
a retention device which retains a machine learning model that has been machine-trained so that, with respect to a plurality of eyes to be examined, at least one of image information of retina and three-dimensional structure information of retina is acquired at each of a plurality of time points, visual field related information relating to a visual field of the eye to be examined is acquired at a corresponding time point, at least one of the image information of the retina and the three-dimensional structure information of the retina regarding each of the eyes to be examined is input to the machine learning model as input information, visual field change information representing change of the visual field and obtained based on the visual field related information at the plurality of time points with regard to the corresponding eye to be examine is used as training information, and visual field change information estimated on the basis of the input information is output from the machine learning model;
an acquisition device which acquires at least one of image information of retina and three-dimensional structure information of retina of an eye to be examined of a person undergoing an eye examination;
an estimation device which inputs the image information of the retina or the three-dimensional structure information of the retina of the eye to be examined of the person undergoing the eye examination, acquired by the acquisition device, to the machine learning model retained in the retention device, and obtains the output from the retention device as an estimation value of change of the visual field related information; and
an output device which outputs the estimation value obtained by the estimation device for subjecting to a predetermined process.

2. A visual field estimation apparatus according to claim 1, wherein either the left eye or the right eye is treated as a learning target eye,
the machine learning model retained in the retention device is machine-trained so that:
with regard to the eye to be examined treated as the learning target eye, the image information of the retina or the three-dimensional structure information of the retina of eye to be examined is input as it is as input information, the visual field change information representing change of the visual field and obtained based on the visual field related information at the plurality of time points with regard to the corresponding eye to be examine is used as training information as it is, and visual field change information estimated on the basis of the input information is output from the machine learning model;
the machine learning model is machine-trained so that:
between the left eye and the right eye, with regard to the eye to be examined which is not treated as the learning target eye, the image information of the retina or the three-dimensional structure information of the retina of the eye to be examined is subjected to right/left inversion and the inverted information is input as input information, the visual field change information representing change of the visual field and obtained based on the visual field related information at the plurality of time points with regard to the corresponding eye to be examine is subjected to right/left inversion and the inverted information is used as training information, and visual field change information estimated on the basis of the input information is output from the machine learning model,
with regard to the eye to be examined treated as the learning target eye, the estimation device inputs the image information of the retina or the three-dimensional structure information of the retina of the eye to be examined to the machine learning model as it is; between the left eye and the right eye, with regard to the eye to be examined which is not treated as the learning target eye, the estimation device applies right/left inversion to the image information of the retina or the three-dimensional structure information of the retina of the eye to be examined, and inputs the information after the right/left inversion to the machine learning model, and
in case that the estimation device outputs the visual field change information with regard to the eye to be examined which is not treated as the learning target eye, the output device applies right/left inversion to the visual field change information and outputs the information after the right/left inversion.

3. A visual field estimation apparatus according to claim 1, wherein
the output device generates information regarding a threshold value of a visual field, a total deviation of a visual field, or a pattern deviation of a visual field, on the basis of the estimation value, and outputs the generated information.

4. A visual field estimation apparatus according to claim 1, further comprising a second machine learning model having been machine-trained to receive at least estimation values obtained by the estimation device with regard to the plurality of eyes to be examined, and information regarding intraocular pressure of the corresponding eye to be examined, as input information, and to output visual field change information estimated on the basis of the input information, wherein, using the second machine learning model,
with regard to the eye to be examined of the person undergoing an eye examination, at least one of the image information of the retina and the three-dimensional structure information of the retina, as well as information regarding intraocular pressure are acquired, and the acquired image information of the retina or three-dimensional structure information of the retina, and the acquired information regarding intraocular pressure, of the eye to be examined of the person undergoing the eye examination are input to the second machine learning model, together with an estimation value obtained by using the machine learning model which is retained in the retention device, and has been machine-trained to receive the image information of the retina or the three-dimensional structure information of the retina as input information, and to output visual field change information estimated on the basis of the input information, and an output from the second machine learning model is treated as an estimation value of change of the visual field related information which is output and is subjected to a predetermined process.

5. A visual field estimation apparatus according to any one of claims 1 to 4, wherein
the visual field change information is information indicating change of a threshold value of the visual field.

6. A visual field estimation apparatus according to any one of claims 1 to 4, wherein
the visual field change information is information indicating change of a predetermined index of the visual field.

7. A method for manufacturing a neural network using a computer, the method comprising:
an acquisition step wherein, with regard to a plurality of eyes to be examined, an acquisition device acquires at least one of image information of retina and three-dimensional structure information of retina at a plurality of time points, and also acquires visual field related information relating to a visual field of each of the eyes to be examined at a corresponding time point,
an input step wherein an input device inputs the image information of the retina or the three-dimensional structure information of the retina with regard to each of the eyes to be examined, to a predetermined machine learning model, as input information, and
a machine learning step wherein a machine learning device subjects a machine learning model to a machine learning processing, using visual field change information representing change of a visual field obtained on the basis of the visual field related information with regard to a corresponding eye to be examined at a plurality of time points, as training information, so that the machine learning model outputs visual field change information estimated on the basis of the input information.

8. A program which functions a computer as:
a retention device which retains a machine learning model that has been machine-trained so that, with respect to a plurality of eyes to be examined, at least one of image information of retina and three-dimensional structure information of retina is acquired at each of a plurality of time points, visual field related information relating to a visual field of the eye to be examined is acquired at a corresponding time point, at least one of the image information of the retina and the three-dimensional structure information of the retina regarding each of the eyes to be examined is input to the machine learning model as input information, visual field change information representing change of the visual field and obtained based on the visual field related information at the plurality of time points with regard to the corresponding eye to be examine is used as training information, and visual field change information estimated on the basis of the input information is output from the machine learning model;
an acquisition device which acquires at least one of image information of retina and three-dimensional structure information of retina of an eye to be examined of a person undergoing an eye examination;
an estimation device which inputs the image information of the retina or the three-dimensional structure information of the retina of the eye to be examined of the person undergoing the eye examination, acquired by the acquisition device, to the machine learning model retained in the retention device, and obtains the output from the retention device as an estimation value of change of the visual field related information; and
an output device which outputs the estimation value obtained by the estimation device for subjecting to a predetermined process.
